(19) United States
(12) Patent Application Publication
(10) Pub. No.: US 2005/0003001 A1
(43) Pub. Date: Jan. 6, 2005

(54) Method for improving dissolution of poorly dispersible medicaments

(76) Inventors: Hisami Yamaguchi, Osaka (JP); Shuji Saka, Osaka (JP); Takao Ueda, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/491,887

(22) PCT Filed: Oct. 30, 2002
(86) PCT No.: PCT/JP02/11315

(30) Foreign Application Priority Data
Nov. 7, 2001 (JP) 2001-314435

(51) Int. Cl. A61K 31/554; A61K 9/20; A61K 9/14

(52) U.S. Cl. 424/464; 514/211.13

(57) ABSTRACT

The present invention is to provide a method for improving the dissolution of a poorly dispersible medicament and that is achieved by preparing a granulated product where a floating agent is added to the poorly dispersible medicament.

### TECHNICAL FIELD

The present invention relates to a method for improving the dissolution of poorly dispersible medicaments and also to a pharmaceutical preparation where the dissolution is improved.

### BACKGROUND ART

When a solid preparation for oral use containing a poorly dispersible medicament is poured into a dissolution test medium, disintegration of the preparation is disturbed due to property changes and crystal transformation of the medicament per se whereby there may be resulted poor dissolution due to aggregation, retention, etc. onto the bottom of the beaker for the dissolution test. With regard to a method for improving a dispersibility of the preparations containing such a poorly dispersible medicament, there has been mostly adopted a method where a large quantity of diluent is compounded with the preparation to reduce the content of the medicament in the preparation whereby the action for disturbing the disintegration due to property changes and crystal transformation is relieved. There has been also adopted a method where granules containing the poorly dispersible medicament are firstly prepared and then a sufficient diluent is added thereto to constitute a preparation whereby the granules containing the poorly dispersible medicament are localized in the preparation so that the disintegration of the preparation as a whole is not delayed. However, according to the methods for improving the poor dispersibility as mentioned above, it is necessary to compound a large quantity of diluent and there is a problem in the case of tablets that the size becomes big and the administration becomes difficult. Further, in the case of fine granules and granules, the amount to be taken by the patient become much as well which will sometimes cause a problem in terms of administration and dispensation.

### BRIEF DESCRIPTION OF DRAWINGS

FIG. 1 is a graph showing the changes in dissolution of each of the granulated products of Test Example 1 into the dissolution medium II of the Japanese Pharmacopoeia with a lapse of time.

FIG. 2 is a graph showing the changes in dissolution of each of the granulated products of Test Example 2 into the dissolution medium II of the Japanese Pharmacopoeia with a lapse of time.

### DISCLOSURE OF THE INVENTION

The present inventors have found that, when granulated product is prepared by adding a floating agent to a poorly dispersible medicament, the poorly dispersible medicament can be floated and its dispersibility can be improved whereby the dissolution of the poorly dispersible medicament is able to be improved. It has been also found that, when the mixing amount of the floating agent is adjusted in that case, dissolution of the poorly dispersible medicament can be adjusted as well.

Thus, in accordance with the present invention, there are provided a method for improving the dissolution of poorly dispersible medicament where the poorly dispersible medicament and a floating agent are contained and the dissolution of the said poorly dispersible medicament is improved by the floating agent, and also granulated products where the dissolution of the poorly dispersible medicament is improved.

The term “poorly dispersible medicament” used in the present invention means a medicament which is not fully dispersed upon pouring into a dissolution test medium because of the reasons that the medicament in a solid state changes to oily or gel-like state or the amorphous medicament is crystallized due to property changes or crystal transformation whereupon the medicament adheres, for example, at the bottom or on the wall of the beaker for the dissolution test.

Examples of the poorly dispersible medicament are 11-[4-[2-(2-hydroxyethoxy)ethyl]-1-piperazinyl]dibenzo[b,f][1,4]thiazepine or a salt thereof and 1-(cyclohexyloxycarbonyloxy)ethyl 7β-[2-(aminothiazol-4-yl)acetamido]-3-[[[1-(2-dimethylaminoethyl)-1H-tetrazol-5-yl]thio]methyl]ceph-3-em-4-carboxylate or a salt thereof. Examples of the salt are fumarate and hydrochloride.

The above 11-[4-[2-(2-hydroxyethoxy)ethyl]- 1-piperazinyl]dibenzo-[b,f][1,4]thiazepine or a salt thereof and 1-(cyclohexyloxycarbonyloxy)ethyl 7β-[2-(aminothiazol-4-yl)acetamido]-3-[[[1-(2-dimethylaminoethyl)-1H-tetrazol-5-yl]thio]methyl]ceph-3-em-4-carboxylate or a salt thereof may be manufactured by the method described in the Japanese Patent Application Publication Nos. 8378/1988 and 218394/1985, respectively.

The term “granulated product” used in the present invention means fine granules, granules, etc. having almost uniform shape and size manufactured not by a mere mixing of powder but by means of artificial operation such as extrusion granulation, tumbling granulation, fluidized bed granulation, dry compression granulation and spray-drying granulation.

The term “fine granules” used in the present invention means a granulated product where the particle size is not more than 850 micrometers in which the particles of 500 micrometers or more are 5% or less and those of 75 micrometers or less are 10% or less, while the term “granules” means a granulated product where the particle size is not more than 1,700 micrometers in which the particles of 1,400 micrometers or more are 5% or less and those of 355 micrometers or less are 15% or less.

Improvement in the dissolution according to the present invention means to increase the dissolution. Adjustment of dissolution means that the improved dissolution can be freely changed within such a range that the dissolution inherent to the poorly dispersible medicament is improved and the adjustment is also included within a coverage of the improvement in dissolution.

The term “floating agent” used in the present invention is a substance which can well disperse a poorly dispersible medicament as a result of floating of the said poorly dispersible medicament when mixed and granulated with the poorly dispersible medicament followed by stirring in a dissolution test medium and its specific examples are non-water-soluble cellulose such as crystalline cellulose, powdery cellulose and low-substituted hydroxypropyl cellulose, sodium alginate, propylene glycol alginate, tragacanth powder and xanthan gum. Particularly preferred one among those exemplified floating agents is crystalline cellulose.

A granulated product in which a floating agent is contained in a poorly dispersible medicament is able to improve the dissolution. Moreover, when it is made into a granulated product containing a surfactant, the granulated product is apt to be disintegrated from its surface due to the surfactant and a fine adjustment of the dissolution can be also carried out easily by adjusting the amounts of the floating agent and the surfactant.

The term “surfactant” used in the present invention is a substance by which disintegration of the granulated substance containing it can be made easy from the surface upon stirring in a dissolution test medium and there may be used common surfactants therefor. Specific examples are polyoxyethylene derivatives of natural fat/oil and wax such as polyoxyethylene stearyl alcohol, polyoxyethylene alkyl ether, polyoxyethylene sorbitan fatty acid ester, polyoxyethylene glyceryl monofatty acid ester, polyoxyethylene propylene glycol monofatty acid ester, polyoxyethylene sorbitol fatty acid ester and polyoxyethylene hydrogenated castor oil; polyethylene glycol fatty acid ester such as polyoxyl 40 stearate; sorbitan fatty acid ester; sucrose fatty acid ester; surfactant of a polyoxyethylene-polyoxypropylene copolymer and block copolymer type such as polyoxyethylene polyoxypropylene glycol; alkyl sulfate salt such as sodium lauryl sulfate; phospholipid; bile acid salt; fatty acid; monohydric alcohol fatty acid ester; ethylene glycol fatty acid ester; and polyhydric alcohol fatty acid ester. Among them, preferred ones are sodium lauryl sulfate, polyoxyl 40 stearate, sucrose fatty acid ester, polyoxyethylene hydrogenated castor oil, polyoxyethylene polyoxypropylene glycol and polyoxyethylene sorbitan fatty acid ester and more preferred one is sodium lauryl sulfate.

The floating agent and the surfactant each may be used solely or two or more of them may be combined.

Compounding ratio of the poorly dispersible medicament in the granulated product of the present invention may be dependent upon the type of the poorly dispersible medicament but, usually, the poorly dispersible medicament is 0.01-0.99 part by weight, preferably 0.05-0.8 part by weight or, more preferably, 0.1-0.7 part by weight to 1 part by weight of the granulated product.

Compounding ratio of the floating agent to the poorly dispersible medicament in the granulated product of the present invention may be dependent upon the type of the poorly dispersible medicament and of the floating agent but, usually, the floating agent is 0.001-10 part(s) by weight, preferably 0.01-1 part by weight or, more preferably, 0.02-0.3 part by weight to 1 part by weight of the poorly dispersible medicament.

Compounding ratio of the surfactant to the poorly dispersible medicament in the granulated product of the present invention may be dependent upon the type of the poorly dispersible medicament, the floating agent and the surfactant but, usually, the surfactant is 0.000001-0.1 part by weight, preferably 0.000005-0.01 part by weight or, more preferably, 0.00002-0.001 part by weight to 1 part by weight of the poorly dispersible medicament.

The granulated product of the present invention may be prepared in such a manner that the starting medicament is pulverized, mixed with various compounding agents in the presence or absence of a suitable solvent, granulated by a conventional granulating method such as extrusion granulation, tumbling granulation, fluidized bed granulation, dry compression granulation and spray-drying granulation, then dried if necessary and made into a uniform size. As to a preferred granulating method, extrusion granulation may be exemplified. In addition to the floating agent or the surfactant, the granulated product may further contain additives which are commonly acceptable for pharmaceuticals such as vehicle, binder, disintegrating agent, sweetener and antistatic agent where those additives may be appropriately selected.

Examples of the vehicle are lactose, starch, white sugar, glucose, mannitol, crystalline cellulose, calcium sulfate and calcium phosphate.

Examples of the binder are ethyl cellulose, methacrylic acid copolymer, gum arabic, polyvinylpyrrolidone, hydroxypropyl cellulose, hydroxypropyl methyl cellulose, carboxymethyl cellulose, gelatin, white sugar, glucose, tragacanth powder and sodium alginate.

Examples of the disintegrating agent are starch, crystalline cellulose, carboxymethyl cellulose calcium, carboxymethyl starch sodium, carboxymethyl cellulose sodium, croscarmellose sodium, crospopidone and low-substituted hydroxypropyl cellulose.

Examples of the sweetener are powdered hydrogenated maltose starch syrup, D-mannitol, aspartame, fructose, lactose, glucose, xylitol and saccharine.

In addition to be used as fine granules and granules, the granulated product of the present invention may also be used in a dosage form such as capsules and tablets containing the granulated product. Those capsules, tablets, etc. may further contain the above-mentioned commonly acceptable additives other than the granulated product such as vehicle, binder, disintegrating agent, sweetener and antistatic agent and such additives may be appropriately selected.

An example of the preferred compounding ratio in the granulated product of the present invention is 0.01-1 part by weight of crystalline cellulose and 0.000005-0.01 part by weight of sodium lauryl sulfate to 1 part by weight of 11-[4-[2-(2-hydroxyethoxy)ethyl]-1-piperazinyl]dibenzo[b,f][1,4]thiazepine fumarate (hereinafter, referred to as “quetiapine fumarate”) and it is more preferred to compound in a ratio of 0.02-0.3 part by weight of crystalline cellulose and 0.00002-0.001 part by weight of sodium lauryl sulfate to 1 part by weight of quetiapine fumarate.

### EXAMPLES

As hereunder, the present invention is illustrated by way of the Examples although the present invention is not limited to those Examples.

### Comparative Example 1

Quetiapine fumarate (230.26 g), 161.74 g of fine powder of lactose and 8 g of hydroxypropyl cellulose were weighed and mixed, 100 mL of 50 vol. % of ethanol were added thereto in a universal mixer/stirrer (type 5DMV manufactured by Sanei Seisakusho) and the mixture was stirred and granulated for 10 minutes (rotating speed: 122 rpm; revolving speed: 58 rpm). This granulated product was transferred to a cylindrical granulator (type HU-G manufactured by Hata Tekkosho) and extruded from pores each having a diameter of 0.5 mm under an operating condition where the rotating speed of expellers was 17 rpm. The product was dried at 40° C. for 17 hours in a ventilating drier and made into a uniform size by sizing through a sieve of 500 μm to give a granulated product satisfying the standard for fine granules stipulated by the Japanese Pharmacopoeia.

### Example 1

Quetiapine fumarate (230.26 g), 141.74 g of fine powder of lactose, 8 g of hydroxypropyl cellulose and 20 g of crystalline cellulose were weighed and mixed and 100 mL of 50 vol. % of ethanol were added followed by subjecting to the same treatment as Comparative Example 1 to give a granulated product satisfying the standard for fine granules stipulated by the Japanese Pharmacopoeia.

### Example 2

Quetiapine fumarate (230.26 g), 121.74 g of fine powder of lactose, 8 g of hydroxypropyl cellulose and 40 g of crystalline cellulose were weighed and mixed and 110 mL of 50 vol. % of ethanol were added followed by subjecting to the same treatment as Comparative Example 1 to give a granulated product satisfying the standard for fine granules stipulated by the Japanese Pharmacopoeia.

### Example 3

Quetiapine fumarate (230.26 g), 81.74 g of fine powder of lactose, 8 g of hydroxypropyl cellulose and 80 g of crystalline cellulose were weighed and mixed and 130 mL of 50 vol. % of ethanol were added followed by subjecting to the same treatment as Comparative Example 1 to give a granulated product satisfying the standard for fine granules stipulated by the Japanese Pharmacopoeia.

### Test Example 1

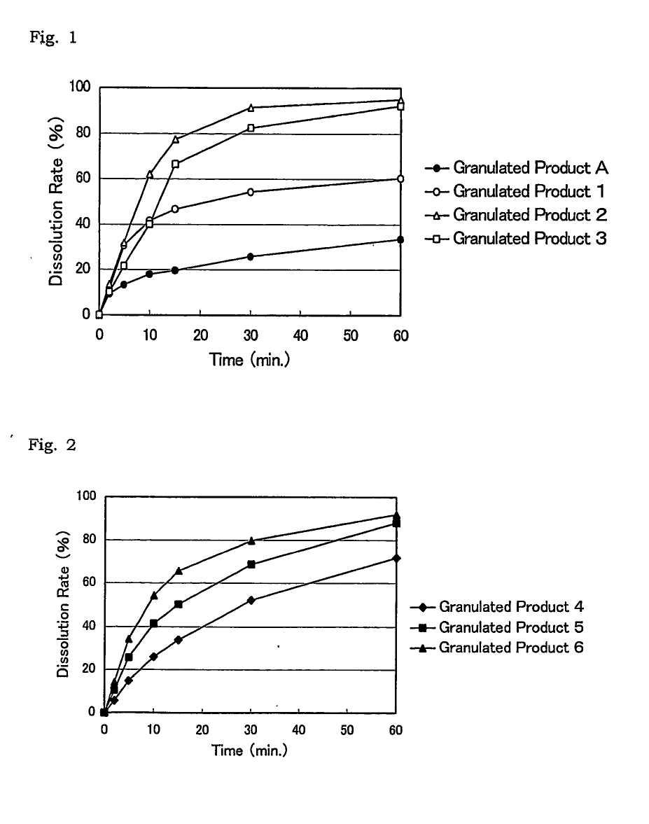

The granulated product prepared in Comparative Example 1 (hereinafter, referred to as “granulated product A”) and those prepared in Examples 1 to 3 (hereinafter, referred to as “granulated product 1”, “granulated product 2” and “granulated product 3”, respectively) were used for comparing the dissolution of quetiapine fumarate (hereinafter, referred to as “the active ingredient”) in each of the preparations. The test was carried out in such a manner that the granulated product containing the active ingredient in an amount corresponding to 25 mg was added to 900 mL of the dissolution medium II of the Japanese Pharmacopoeia, warmed at 37° C. and stirred at 50 rpm of the paddle rotations to determine the concentration of the active ingredient with a lapse of time. The result is shown in FIG. 1. As will be apparent from FIG. 1, dissolution of the active ingredient was improved by crystalline cellulose and the dissolution was able to be adjusted by the content of the crystalline cellulose.

### Example 4

Quetiapine fumarate (230.26 g), 54.35 g of fine powder of lactose, 8 g of hydroxypropyl cellulose, 20 g of crystalline cellulose, 60 g of partly pregelatinized starch, 48 g of xylitol and 12 g of aspartame were weighed and mixed and 130 mL of 50 vol. % of ethanol were added followed by subjecting to the same treatment as Comparative Example 1 to give a granulated product satisfying the standard for fine granules stipulated by the Japanese Pharmacopoeia.

### Example 5

Quetiapine fumarate (230.26 g), 54.338 g of fine powder of lactose, 8 g of hydroxypropyl cellulose, 20 g of crystalline cellulose, 60 g of partly pregelatinized starch, 48 g of xylitol and 12 g of aspartame were weighed and mixed and 130 mL of 50 vol. % of ethanol wherein 0.012 g of sodium lauryl sulfate was dissolved were added followed by subjecting to the same treatment as Comparative Example 1 to give a granulated product satisfying the standard for fine granules stipulated by the Japanese Pharmacopoeia.

### Example 6

Quetiapine fumarate (230.26 g), 54.23 g of fine powder of lactose, 8 g of hydroxypropyl cellulose, 20 g of crystalline cellulose, 60 g of partly pregelatinized starch, 48 g of xylitol and 12 g of aspartame were weighed and mixed and 130 mL of 50 vol. % of ethanol wherein 0.12 g of sodium lauryl sulfate was dissolved were added followed by subjecting to the same treatment as Comparative Example 1 to give a granulated product satisfying the standard for fine granules stipulated by the Japanese Pharmacopoeia.

### Test Example 2

The granulated products prepared in Examples 4 to 6 (hereinafter, referred to as “granulated product 4”, “granulated product 5” and “granulated product 6”, respectively) were used for comparing the dissolution of quetiapine fumarate (hereinafter, referred to as “the active ingredient”) in each of the preparations. The test was carried out in such a manner that the granulated product containing the active ingredient in an amount corresponding to 25 mg was added to 900 mL of the dissolution medium II of the Japanese Pharmacopoeia, warmed at 37° C. and stirred at 50 rpm of the paddle rotations to determine the concentration of the active ingredient with a lapse of time. The result is shown in FIG. 2. As will be apparent from FIG. 2, dissolution of the active ingredient was able to be adjusted by the content of the sodium lauryl sulfate.

### Example 7

Quetiapine fumarate (345.39 g), 498.81 g of fine powder of lactose, 20 g of hydroxypropyl cellulose, 50 g of crystalline cellulose, 50 g of partly pregelatinized starch and 35 g of aspartame were weighed and mixed and 290 mL of 50 vol. % of ethanol wherein 0.3 g of sodium lauryl sulfate was dissolved were added followed by subjecting to the same treatment as Comparative Example 1 to granulate. To 900 g of the granules was added 0.45 g of hydrated silicon dioxide to give a granulated product satisfying the standard for fine granules stipulated by the Japanese Pharmacopoeia.

### Example 8

Quetiapine fumarate (345.39 g), 383.81 g of fine powder of lactose, 20 g of hydroxypropyl cellulose, 50 g of crystalline cellulose and 200 g of powdered hydrogenated maltose starch syrup were weighed and mixed and 290 mL of 50 vol. % of ethanol wherein 0.3 g of sodium lauryl sulfate was dissolved were added followed by subjecting to the same treatment as Comparative Example 1 to granulate. To 900 g of the granules was added 0.45 g of hydrated silicon dioxide to give a granulated product satisfying the standard for fine granules stipulated by the Japanese Pharmacopoeia.

### Example 9

Quetiapine fumarate (345.39 g), 534.11 g of D-mannitol, 20 g of hydroxypropyl cellulose and 100 g of crystalline cellulose were weighed and mixed and 290 mL of 50 vol. % of ethanol were added followed by subjecting to the same treatment as Comparative Example 1 to granulate. To 900 g of the granules was added 0.45 g of hydrated silicon dioxide to give a granulated product satisfying the standard for fine granules stipulated by the Japanese Pharmacopoeia.

### Example 10

1-(Cyclohexyloxycarbonyloxy)ethyl 7β-[2-(aminothiazol-4-yl)acetamido]-3-[[[1-(2-dimethylaminoethyl)-1H-tetrazol-5-yl]thio]methyl]ceph-3-em-4-carboxylate hydrochloride (hereinafter, referred to as “cefotiam hexetil hydrochloride”) (230.26 g), 121.74 g of fine powder of lactose, 8 g of hydroxypropyl cellulose and 40 g of crystalline cellulose were weighed and mixed and 110 mL of 50 vol. % of ethanol were added followed by subjecting to the same treatment as Comparative Example 1 to give a granulated product satisfying the standard for fine granules stipulated by the Japanese Pharmacopoeia.

### Example 11

Cefotiam hexetil hydrochloride (230.26 g), 54.23 g of fine powder of lactose, 8 g of hydroxypropyl cellulose, 20 g of crystalline cellulose, 60 g of partly pregelatinized starch, 48 g of xylitol and 12 g of aspartame were weighed and mixed and 130 mL of 50 vol. % of ethanol wherein 0.12 g of sodium lauryl sulfate was dissolved were added followed by subjecting to the same treatment as Comparative Example 1 to give a granulated product satisfying the standard for fine granules stipulated by the Japanese Pharmacopoeia.

1-18. (Canceled).

19. A granulated product where dissolution of a poorly dispersible medicament is improved, which comprises the poorly dispersible medicament and a floating agent, wherein the dissolution of the poorly dispersible medicament is improved by the floating agent.

20. The granulated product according to claim 19, which further comprises a surfactant.

21. The granulated product according to claim 20, wherein the granulated product is manufactured by an extrusion granulation.

22. The granulated product according to claim 20, wherein the granulated product is in a form of fine granules.

23. A capsule which comprises the granulated product described in claim 20.

24. A tablet which comprises the granulated product described in claim 20.

25. The granulated product according to claim 20, wherein the floating agent is non-water-soluble cellulose, sodium alginate, propylene glycol alginate, tragacanth powder or xanthan gum.

26. The granulated product according to claim 20, wherein the surfactant is sodium lauryl sulfate, polyoxyl 40 stearate, sucrose fatty acid ester, polyoxyethylene hydrogenated castor oil, polyoxyethylene polyoxypropylene glycol or polyoxyethylene sorbitan fatty acid ester.

27. The granulated product according to claim 20, wherein the poorly dispersible medicament is 11-[4-[2-(2-hydroxyethoxy)ethyl]-1-piperazinyl]-dibenzo[b,f][1,4]thiazepine or a salt thereof.

28. The granulated product according to claim 27, wherein the floating agent is crystalline cellulose and the surfactant is sodium lauryl sulfate.

29. The granulated product according to claim 28, wherein the granulated product is manufactured by an extrusion granulation.

30. The granulated product according to claim 28, wherein the granulated product is in a form of fine granules.
